(19) United States
(12) Patent Application Publication
(10) Pub. No.: US 2020/0303046 A1
(43) Pub. Date: Sep. 24, 2020

(54) WI-FI-BASED CONDITION MONITORING

(71) Applicant: Aerial Technologies Inc., Montreal (CA)

(72) Inventors: Michel Allegue Martinez, Terrebonne (CA); Negar Ghourchian, Montreal (CA); David Grant, Santa Rosa Valley, CA (US); Francois Morel, Kirkland (CA); Pascal Paradis-Theberge, Montreal (CA)

(21) Appl. No.: 16/798,319

(22) Filed: Feb. 22, 2020

Related U.S. Application Data
(60) Provisional application No. 62/809,380, filed on Feb. 22, 2019.
(60) Provisional application No. 62/809,406, filed on Feb. 22, 2019.

(51) Int. Cl. G16H 10/60; G16H 40/60; H04W 8/22

(52) U.S. Cl. CPC G16H 10/60; H04W 84/12; H04W 8/22; G16H 40/60

(57) ABSTRACT

Technologies for attestation techniques, systems, and methods are provided for determining patient movement and conditions. The patient's movement may be determined by passive indoor positioning technology using channel state information (CSI). Activity data from an activity database may be retrieved from a Wi-Fi access point located in proximity to a monitored space and analyzed to determine an activity trend of a patient in the monitored space. A electronic medical records database may be polled for one or more health events each associated with a set of health parameters. The activity data may be compared to the associated parameter sets of the health events to identify any matches. A notification may be sent to a designated device (e.g., of a caregiver) based on a match between the activity data or trends thereof with the parameter set of a health event,

### CROSS-REFERENCE TO RELATED APPLICATIONS

The present application claims the priority benefit of U.S. Provisional Patent Application No. 62/809,380 filed on Feb. 22, 2019 and entitled “Patient Monitoring,” and U.S. Provisional Patent Application No. 62/809,406 filed on Feb. 22, 2019 and entitled “Wi-F-Based Condition Monitoring,” the disclosures of which are incorporated herein by reference in their entirety.

### BACKGROUND OF THE INVENTION

### 1. Field of the Invention

The present disclosure is generally related to monitoring individual conditions, and more specifically to using passive Wi-Fi-based motion detection systems to trach movement and conditions of patients.

### 2. Description of the Related Art

Motion detection is the process of detecting a change in the position of an object relative to its surroundings or a change in the surroundings relative to an object. Motion detection can be accomplished by a software-based monitoring algorithm, which, for example when it detects motions may signal the surveillance camera to begin capturing the event. An advanced motion detection surveillance system can analyze the type of motion to see if it warrants an alarm.

It is desirable to have an integrated and wireless means of detecting individuals and their actions or inactions as well as determining the nature of the actions or inactions.

### SUMMARY OF THE CLAIMED INVENTION

Disclosed herein are systems, methods, and computer-readable storage media for determining patient movement. The patient's movement may be determined by passive indoor positioning technology using channel state information (CSI). In some aspects, an exemplary method can include analyzing activity data from an activity database regarding Wi-Fi access point localization to determine an activity trend of a patient in the monitored space; polling a electronic medical records database for one or more health events having associated parameters associated with the patient, comparing the activity data with the associated parameters of the one or more health events, and sending a first notification to a device associated with a caregiver when the activity data are within the associated parameters. The movement of the patient may be translated into the activity data and determined using channel state information (CSI).

### BRIEF DESCRIPTION OF THE FIGURES

FIG. 1A illustrates an exemplary network environment in which a system for Wi-Fi-based monitoring of individual conditions may be implemented.

FIG. 1B illustrates an exemplary health events database.

FIG. 1C illustrates an exemplary activity database.

FIG. 1D illustrates an exemplary notification database.

FIG. 1E illustrates an exemplary electronic medical records database.

FIG. 1F illustrates an exemplary caregiver event database.

FIG. 1G illustrates an exemplary activity safety threshold activity database.

FIG. 1H illustrates an exemplary threshold database.

FIG. 2 is a flowchart illustrating an exemplary method for Wi-Fi-based monitoring of individual conditions.

FIG. 3 is a flowchart illustrating an exemplary method for activity analysis.

FIG. 4 is a flowchart illustrating an exemplary method for determining health indications.

FIG. 5 is a flowchart illustrating an exemplary method for notification generation.

FIG. 6 is a flowchart illustrating an exemplary method for updating caregivers.

FIG. 7 is a flowchart illustrating an exemplary method for evaluating safety thresholds.

FIG. 8 is a flowchart illustrating an exemplary method for condition analysis.

### DETAILED DESCRIPTION

Disclosed herein are systems, methods, and computer-readable storage media for determining patient movement. The patient's movement may be determined by passive indoor positioning technology using channel state information (CSI). In some aspects, an exemplary method can include analyzing activity data from an activity database regarding Wi-Fi access point localization to determine an activity trend of a patient in the monitored space; polling a electronic medical records database for one or more health events having associated parameters associated with the patient, comparing the activity data with the associated parameters of the one or more health events, and sending a first notification to a device associated with a caregiver when the activity data are within the associated parameters. The movement of the patient may be translated into the activity data and determined using channel state information (CSI).

The exemplary method may further include polling a caregiver database for custom parameters of the one or more health events; comparing the activity data with the custom parameters; and sending a second notification to the device associated with the caregiver when the activity data are within the custom parameters.

The exemplary method may further include receiving, from a user interface of the device associated with the caregiver, updates to the custom parameters. The example method may further include: polling a threshold database for threshold parameters for activity criterion, location change criterion, and time criterion; comparing the activity data with the threshold parameters; and sending an alert effector action to an alert effector when the activity data are within the threshold parameters.

The one or more health events may be one of the following: a diagnosis, a health condition, a procedure, or an operation. The associated parameters may be for average rate of trips to a bathroom, sleep time, and active movement. The activity data may be associated with a radio map using Wi-Fi localization to translate Wi-Fi signal strengths into locations and movement. The radio map may further comprise metadata including frequency data of a channel, phase response data of the channel, and impulse response data of the channel that describe a wireless communication link between paired devices used to compare with a signal scan.

The technologies herein can provide patient location and movements based upon a device-free indoor positioning technology that can monitor patients in a monitored space based on passively observing changes in the environment. Such changes could be determined based on comparisons of types of movements that have built an ensemble of fingerprints during a training phase. During a testing phase, fingerprints generated from new data could be compared with those from the training phase to determine a location or types of movements of an individual. The differences between the fingerprints generated from the new data and those from the training phase could result in data indicating position or engagement of movement, as well as reflecting where and how much an individual is moving in the monitored space.

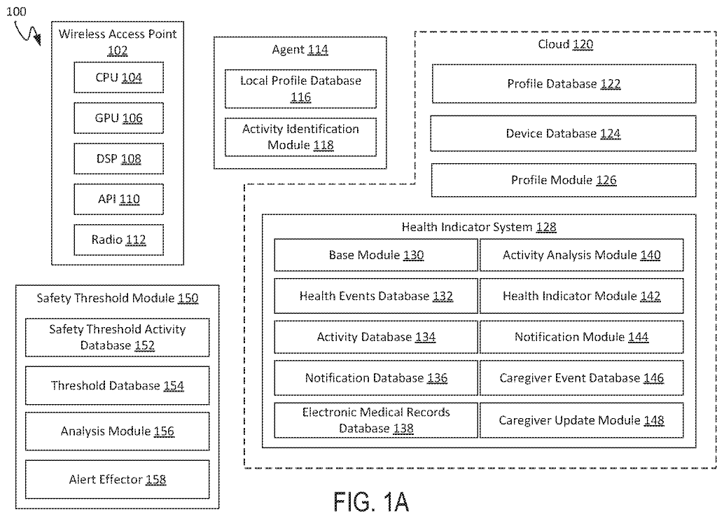

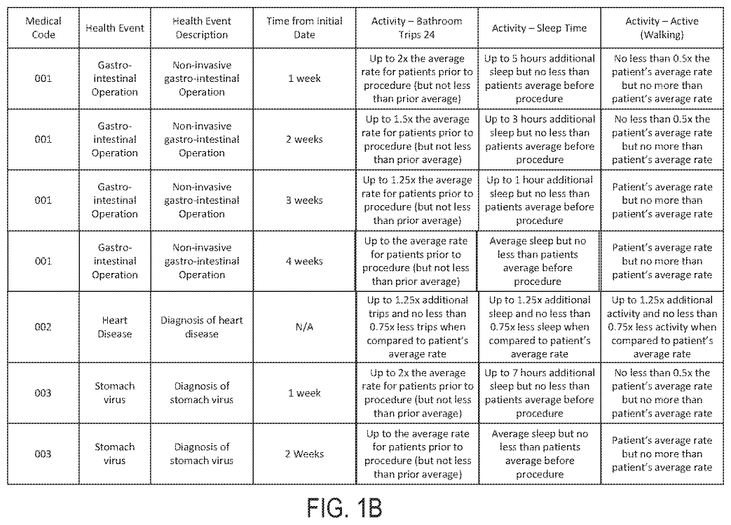

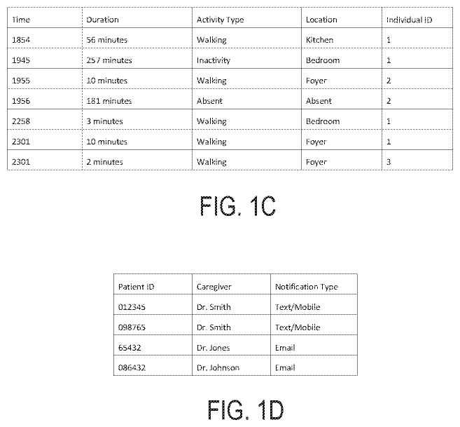

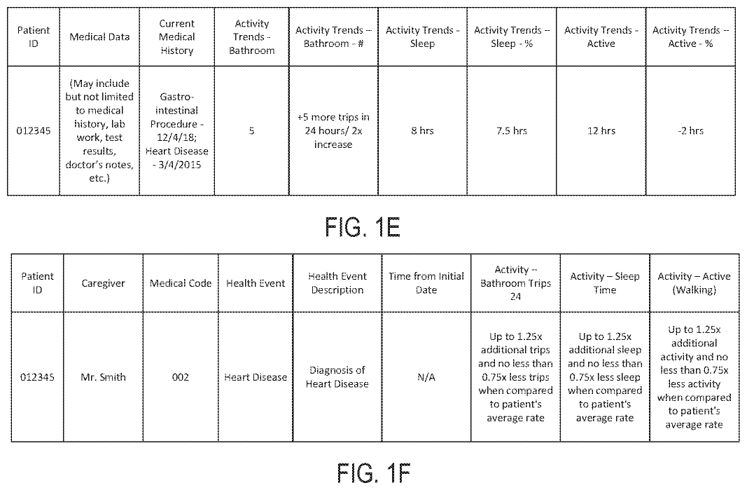

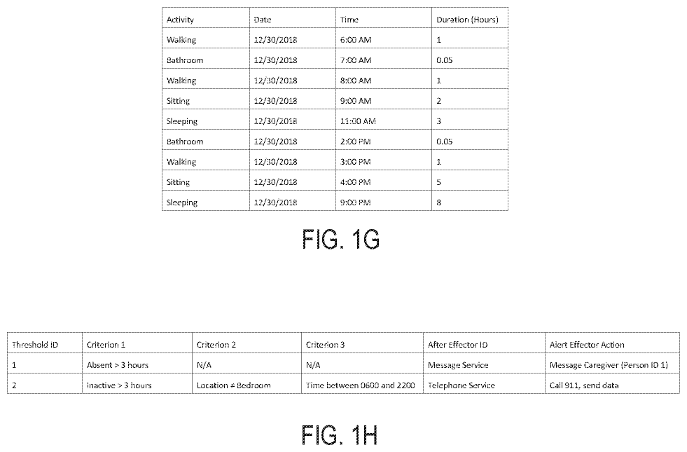

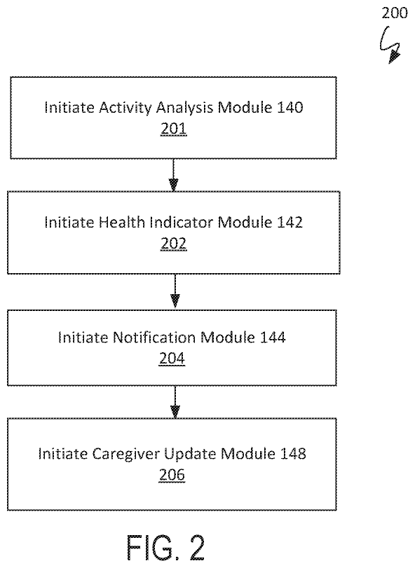

The approaches herein can provide systems, methods, and computer-readable storage media for determining patient movement, wherein the patient's movement is determined by passive indoor positioning technology using channel state information (CSI). The disclosure begins with an initial discussion of systems and technologies for determining patient movement through the passive indoor positioning technology using CSI, as generally illustrated in an exemplary network environment 100 of FIG. 1A. FIGS. 1B through 1F, as well as FIGS. 9A and 9B, illustrate exemplary databases of the network environment 100. FIGS. 2 through 8 illustrate exemplary flows with respect to modules for the network environment 100.

The disclosure now turns to an overview regarding the wireless communication technology for location-based services for patient monitoring.

FIG. 1A illustrates an exemplary network environment 100 in which a system for Wi-Fi-based monitoring of individual conditions may be implemented. The exemplary network environment 100 may be a device-free localization and activity monitoring system in a smart indoor environment that further uses machine-learning algorithms to learn and recognize various kinds of activities. The exemplary network environment 100 may comprise a Wi-Fi access point (AP) 102 configured to record channel state information (CSI). In Wi-Fi communications, CSI refers to known channel properties of a radio frequency (RF) communication link that describes how a signal propagates from a transmitter to a receiver and represents a combined effect of various properties such as channel frequency response data, channel phase response data, and/or channel impulse response data. The frequency response data may be associated with a radio map using Wi-Fi localization to translate Wi-Fi signal strengths into locations. The radio map may further comprise metadata including frequency data of a channel, phase response data of the channel, and impulse response data of the channel that describe a wireless communication link between paired devices used to compare with a signal scan.

The Wi-Fi AP 102 may comprise a central processing unit (CPU) 104 that carries out instructions for the Wi-Fi AP 102 to perform. The Wi-Fi AP 102 may also comprise a graphics processing unit (GPU) 106 that is a specialized electronic circuit designed to rapidly manipulate and alter memory to accelerate the creation of images in a frame buffer intended for output to a display device. The Wi-Fi AP 102 may also comprise a digital signal processor (DSP) 108 that is a specialized microprocessor, or a system-in-a-package (SiP) component, with its architecture optimized for the operational needs of digital signal processing. The DSP 108 may be configured to measure, filter, or compress continuous real-world analog signals. The Wi-Fi AP 102 may also comprise an application program interface (API) 110 that is a set of routines, protocols, and tools for building software applications, programming any graphical user interface (GUI) components, and specifying how software components interact. The API 110 may provide metadata related to the CSI to an agent 114.

The Wi-Fi AP 102 may comprise a radio component 112 that is compliant with either 802.11b or 802.11g, using a stock omnidirectional antenna, and may have a range of 100 m (0.062 mi). The radio component 112 may have an external semi-parabolic antenna (15 dB gain) with a similarly equipped receiver at the far end might have a range over 20 miles. The Wi-Fi AP 102 may be equipped with a network interface card (NIC) that connects the Wi-Fi AP 102 to a computer network. The radio component 112 may be a transceiver, a transmitter, or a receiver.

The agent 114 may collect CSI-related metadata from the Wi-Fi AP 102, filter the CSI-related metadata, and send the filtered CSI-related metadata to one or more cloud server 120 for activity identification. The activity identification can be accomplished on an edge, at an agent level, or in the cloud, or some combination of the three. The agent 114 may comprise a local profile database 116 that is utilized when at least a portion of the activity identification is done on the edge. This could be a simple motion versus no-motion determination profile database or a more extensive profile database used for identifying activities, objects, individuals, biometrics, etc.

The agent 114 may also comprise an activity identification module 118 that distinguishes between activities, such as between walking and in-place activities. In general, a walking activity may cause significant pattern changes to amplitude over time of the channel impulse response, since it involves significant body movements and location changes. In contrast, an in-place activity (such as watching TV on a sofa) only involves relative smaller body movements and may not cause significant pattern changes to amplitude over time of the channel impulse response. Instead, in-place activity is reflected by certain repetitive patterns within the channel impulse response. Wi-Fi localization information related to the motion data can, in some examples, be used to help quantify the level of engagement motion.

The one or more cloud servers 120 may also analyze and create profiles describing various activities. The one or more cloud servers 120 may comprise a profile module 126 that monitors the received CSI-related metadata from a continuous monitoring of a monitored space. The profile module 126 may also identify multiple similar patterns of a set of CSI-related metadata that do not have a matching profile in a profile database 122 of the one or more cloud servers 120. Then, the profile module may combine the set of CSI-related metadata with a user feedback to label the resulting clusters to define a new profile that is then added to the profile database 122. The profiles in the profile database 122 may be simple motion versus no-motion determination profiles or a more extensive profile database used for identifying activities, objects, individuals, biometrics, etc. The one or more cloud servers 120 may further comprise a device database 124 that stores device ID of all connected Wi-Fi APs.

A health indicator system 128 may run on the one or more cloud servers 120 and/or on a remote server. The health indicator system 128 determines if there is a change in a user or patient's health by comparing user or patient activity data from the Wi-Fi AP 102 and compares it to heath data to determine if there is a change in the user or patient's health. A base module 130 stores data from the agent 114, i.e. activity data in the activity database for a user, and then initiates an activity analysis module 140. The activity analysis module 140 may then call a health indicator module 142, a notification module 144, and/or a caregiver update module 148.

The activity analysis module 140 analyzes activity data from an activity database 134 of the health indicator system 128 by comparing the activity data and changes thereto with respect to an estimated model or a set of collected statistics for a user or patient. For example, the activity analysis module 140 may calculate a regular user behavior for trips a user takes to the bathroom in 24 hours and note this in an electronic medical record database 138. As the activity analysis module 140 continues to monitor the user's activity it may calculate changes in the user's regular behavior for trips to the bathroom and make an indication of the change in the electronic medical record database 138.

The health indicator module 142 polls the electronic medical records database 138 for any major health events. The health indicator module 142 uses a health events database 132 to identify the health events. The health indicator module 142 then compares the patient's activity data created by the activity analysis module 140 with activity metrics for the health event found in the health event database 132. For example, the health indicator module 142 may identify that the patient had an operation 4 weeks ago and there are several parameters for activity for the patient's recovery. One of those parameters or metrics may be how close the patient should be to their regular or known behavior for trips to the bathroom. If the patient is not within the parameters, the data is added to the electronic medical record database 138 and the patient's caregiver is notified through the notification module 144.

Furthermore, if the health indicator module 142 does not find any health events that match the health event database 132, the health indicator module 142 may default to “No Event” parameters and metric. “No Event” parameters and metrics could be associated with normal range for different activities for a patient. The purpose is to identify any changes in a person's health that may not directly relate to a major health event. The notification module 144 is initiated when the health indicator module 142 determines that a patient's activity is outside the normal range for a health event and is used to notify the patient's caregiver.

The network environment 100 may also comprise a safety threshold module 150 that stores data from the activity identification module 118 and compares the results of analysis of that data with pre-stored thresholds. If a threshold is exceeded, one or more alert effector 158 is initiated as instructed by in a threshold database 154. The safety threshold module 150 may also comprise a threshold safety activity database 152 that contains stored activity/inactivity data as determined by the agent 114 and activity identification module 118. Such data may include activity or inactivity types, time and location data for determined activity or inactivity, individual identification data, and any other data which may be used by the system.

The threshold database 154 may contain stored activity/inactivity data and thresholds, which may be compared to data saved in the threshold safety activity database 152 or determined by agent 114, for example, the amount of time since a user was active or was within detection range of the Wi-Fi AP 102. Each threshold should have, associated with it, one or more actions to be taken by one or more alert effectors 158 if the threshold is exceeded. An analysis module 156 of the safety threshold module 150 may be one or more computer programs that calculate one or more values from the data stored in an safety threshold activity database 152 and compares that data to threshold data stored in the threshold database 154. If a threshold is exceeded, the action associated with that threshold is sent to the one or more alert effectors 158 for effectuation. The one or more alert effectors 158 may be one or more devices that create an effect, such as a communications device, and audio or visual alarm, a broadcaster to a tracking device, etc.

FIG. 1B illustrates an exemplary health events database (e.g., health events database 132), which stores data with various health events. A health event could include, but not limited to, a diagnosis or health condition, a procedure, an operation, etc., if they occur over a set duration, indicate a potential health problem. Each of these health events could have associated with its certain activity parameters or metrics. The purpose of the activity parameters or metrics suggests normal ranges for the health event. For example, a patient who has an operation could be expecting to perform a certain amount of different activities over time. Activity that it outside the expected parameters may suggest the patient is not recovering as expected. With respect to a health condition, a patient with a specific health condition could be expected to have activities in a certain range. If activity, such as regular or known behavior of trips to the bathroom are outside the parameters or metrics it could indicate that the health condition is getting worse or improving.

FIG. 1C illustrates an exemplary activity database (e.g., activity database 134) that stores all of the activities of a patient or user. The data is collected from the agent 114 and contains a description of the activity, the date of the activity, the time and duration of the activity.

FIG. 1D illustrates an exemplary notification database (e.g., notification database 136), which stores information for notifying a caregiver for a patient, in accordance with some implementations. FIG. 1E illustrates an exemplary electronic medical records database (e.g., electronic medical records database 138), which may store a variety of patient medical data, including but not limited to, medical history, lab work, test results, doctor notes, diagnosis codes, patient activity data, fitness tracker data, etc., in accordance with some implementations.

FIG. 1F illustrates an exemplary caregiver event database (e.g., caregiver event database 146), which stores similar data to the health event database 132 but is specific to a patient and is customized by the caregivers, in accordance with some implementations. For example, a caregiver may change, either increase or decrease, the metrics for a patient's regular or known behavior of trips to the bathroom after being notified by the notification module the first time. When the health indicator module 142 sees that the patient is still outside the health event metrics, the health indicator module 142 may check the caregiver event database 146 so that the caregiver isn't notified again for an issue they have already address unless it gets worse or improves. The caregiver update module 148 allows the caregiver to create customized health event parameters or metrics for their patients after being notified.

The caregiver event database 146 stores similar data to the health event database 132 but is specific to a patient and is customized by the caregiver. For example, a caregiver may change, either increase or decrease, the metrics for a patient's regular or known behavior for trips to the bathroom after being notified by the notification module the first time. When the health indicator module 142 see that the patient is still outside the health event metrics it may check to caregiver event database 146 so that the caregiver isn't notified again for an issue they have already address unless it gets worse or improves.

FIG. 1G illustrates an exemplary activity safety threshold activity database (e.g., threshold safety activity database 152), which stores information for notifying a caregiver for a patient, in accordance with some implementations. The threshold safety activity database 152 may contain stored activity/inactivity data as determined by the agent 114 and activity identification module 118. Such data may include activity or inactivity types, time and location data for determined activity or inactivity, individual identification data, and any other data which may be used by the system.

FIG. 1H illustrates an exemplary threshold database (e.g., threshold database 154), which stores information for notifying a caregiver for a patient, in accordance with some implementations. The threshold database 154 contains stored activity/inactivity data and thresholds that may be compared to data saved in the threshold safety activity database 152 or determined by agent 114, for example, the amount of time since a user was active or was within detection range of the Wi-Fi AP 102. Each threshold should have, associated with it, one or more actions to be taken by one or more alert effectors 158 if the threshold is exceeded.

FIG. 2 is a flowchart illustrating an exemplary method 200 for Wi-Fi-based monitoring of individual conditions. Method 200 may be performed by executing the base module 130 of the health indicator system 128. One skilled in the art may appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The process begins at step 201 with initiating the activity analysis module 140, which analyzes activity data in the activity database 134 and calculating for regular or known behaviors for a user or patient as well as any changes in activity. For example, the activity analysis module 140 may calculate a regular or known behavior for trips a user takes to the bathroom in 24 hours and note this in the electronic medical records database 138. As the activity analysis module 140 continues to look at the user's activity, the activity analysis module 140 may calculate changes in the user's regular or known behavior for trips to the bathroom and make an indication of the change in the electronic medical records database 138.

At step 202, the health indicator module 142 is initiated and polls a user's or patient's electronic medical records database 138 for any major health events. The health indicator module 142 may use the health events database 132 to identify the health events. The health indicator module 142 may then compare the patient's activity data created by the activity analysis module 140 with activity metrics for the health event found in the health event database 132. For example, the health indicator module 142 may identify that the patient had an operation 4 weeks ago and there are several parameters for activity for the patient's recovery. One of those parameters or metrics may be how close the patient should be to their regular or known behavior of trips to the bathroom. If the patient isn't within the parameters, the data is added to the electronic medical record database 138 and the patient's caregiver is notified through the notification module 144. Furthermore, if the module doesn't find any health events that match the health event database 132, the module may default to “No Event” parameters and metric which could be the normal range for different activities for a patient. The purpose of this is to identify any changes in a person's health that may not directly relate to a major health event.

At step 204, a notification module 144 is initiated when the health indicator module 142 determines that a patient's activity is outside the normal range for a health event and is used to notify the patient's caregiver. At step 206, a caregiver update module 148 is initiated to allow the caregiver to create customized health event parameters or metrics for their patients after being notified.

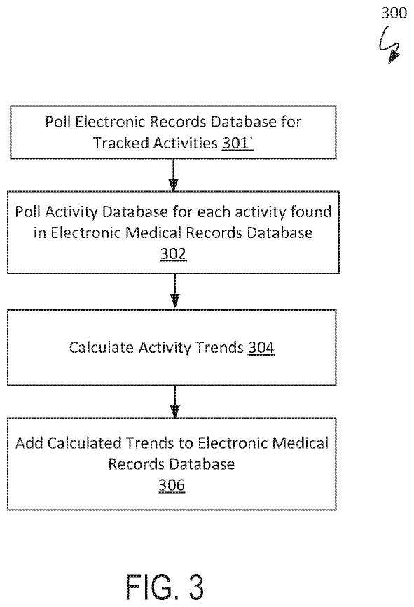

FIG. 3 is a flowchart illustrating an exemplary method 300 for activity analysis. Method 300 may be performed by executing the activity analysis module 140 of the health indicator system 128. The process begins at step 301 with the activity analysis module 140 polling the electronic medical records database 138 for a list of activities. These activities may include, but are not limited to, number of trips to the bathroom, time slept, time active during the day, and time not active during the day. At step 302, the activity analysis module 140 then polls activity database 134 for each of the activities found in the electronic medical records database 138. For each activity the module then calculates a set of trends based on the activity data in the activity database 134. The module may calculate change in activity over certain periods of time such as over a 24-hour period, a week, a month and a year. The module could further calculate a percentage change in a user's regular or known behavior of activity over time. The calculation can be customized based on the activity and/or the way the data may be used in the electronic medical records at step 304. Once the trends for all the activities have been calculated, the module write or stores the calculations to the electronic medical records database 138 at step 306.

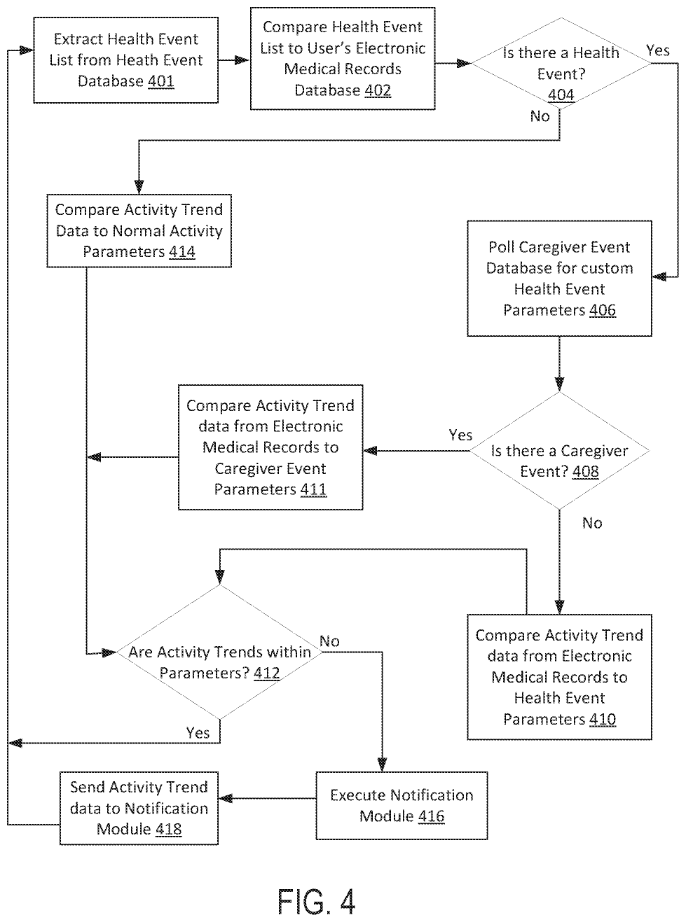

FIG. 4 is a flowchart illustrating an exemplary method 400 for determining health indications. Method 400 may be performed by executing the health indicator module 142 of the health indicator system 128. The process begins at step 401 with the health indicator module 142 first extracting a list of health events from the health event database 132. This list is a pre-populated list of possible health events such as health conditions, procedures, or operations. The health indicator module 142 then compares the list of health events to a user's or patient's electronic medical records at step 402. The health indicator module 142 compares the data to see if there is a health event listed in the electronic medical records database 138 at step 404.

If there are no health events in the patient's medical record, the health indicator module 142 can still check to ensure that the patent is still with in normal activity level at step 405. The health event database 132 stores a profile for normal trends and if no health event is present, the health indicator module 142 compares the activity trends to the normal parameters.

If there is a match to a health event, the health indicator module 142 checks the caregiver event database 146 to check if a caregiver has set custom parameters for the patient at step 406. The electronic medical records database 138 may include a section that lists current medical conditions or procedures for a patient or user. These medical conditions may have an onset date or date of the procedure. For example, a patient may have had a non-invasive gastrointestinal procedure 4 weeks ago, which could be listed in the medical record and the health indicator module 142 could find a match with the health event as there could be a possible recovery parameter for the procedure.

For example, a patient that had a non-invasive gastrointestinal procedure who had a previous similar procedure and took longer to recover, the caregiver may increase the recovery parameters so as not to get false alarms as it is know that the patient may take longer to recover. The health indicator module 142 then determines if there was a match with the caregiver event database 146 and the health event for the patient at step 408. If there is no match with the caregiver event database 146, the activity trends from the patient's electronic medical records database 138 are compared to the health event parameters at step 410. . If there is a match, the activity trends from the patient's electronic medical records database 138 are compared to the caregiver event parameters at step 411.

The health indicator module 142 then determines if the activity trends are within or outside of the event parameters at step 412. If the activity trends are outside the event parameters, the notification module 144 is executed to notify the caregiver of the change in activity at step 416. The activity trends and event parameters that were compared are sent to the notification module at step 418. If the activity trends are within the event parameters, then no notifications are sent to the caregiver and the health indicator module 142 runs from step 401 again.

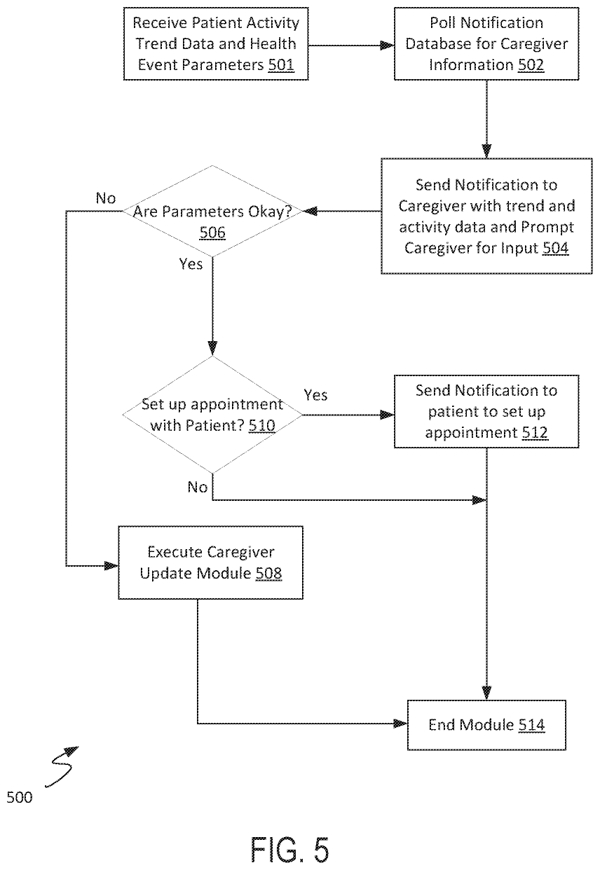

FIG. 5 is a flowchart illustrating an exemplary method 500 for notification generation. Method 500 may be performed by executing the notification module 144 of the health indicator system 128. The process begins with the notification module 144 receiving the patient activity trend data and health event parameters from the health indicator module 142 at step 501. The health event parameters maybe either the parameters from the health event database 132 or caregiver event database 146 depending on which parameters were used by the health indicator module 142. The notification database 136 may then be polled to find the caregiver and means of notification at step 502. A notification is then sent to the caregiver with the activity trend data and the event parameters at step 504.

At the same time the caregiver is prompted for input if the current parameters are still okay for the patient or if they should be updated at step 506, wherein the caregiver is asked if the event parameters are still appropriate for the patient. If the caregiver doesn't think the parameters are appropriate, they can update the parameters and the caregiver update module 148 is then executed at step 508. If the parameters are still appropriate, the caregiver is asked if they want to notify the patient to set up an appointment at step 510.

If the caregiver wants an appointment based on the data, the patient is sent a notification to set up an appointment at step 512. If the caregiver does not want an appointment the module ends at step 514. There are a number of reason a caregiver may not want an appointment with a patient. For example, the caregiver may think it is not a serious issue at the moment and not need to see the patient, or if the caregiver just want to monitor the activity to see if it continues to change, or it's possible the caregiver may plan on seeing the patient already.

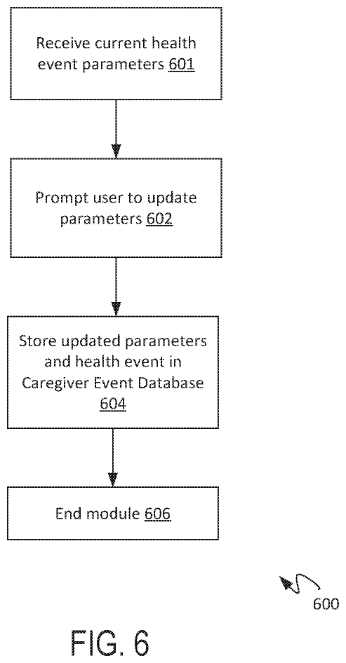

FIG. 6 is a flowchart illustrating an exemplary method 600 for updating caregivers. Method 600 may be performed by executing the caregiver update module 148 of the health indicator system 128. The process begins with the caregiver update module 148 is executed by the notification module 144 and receiving the current health event parameters at step 601. The caregiver is then prompted to update the parameters via a user interface at step 602. For example, a parameter for the number of trips to the bathroom in a 24-hour period maybe 5 but the caregiver may update that to 10 based on their knowledge and history of the patient. The updated parameters are then stored in the caregiver event database 146 at step 604. Once the data is stored the module ends at step 606.

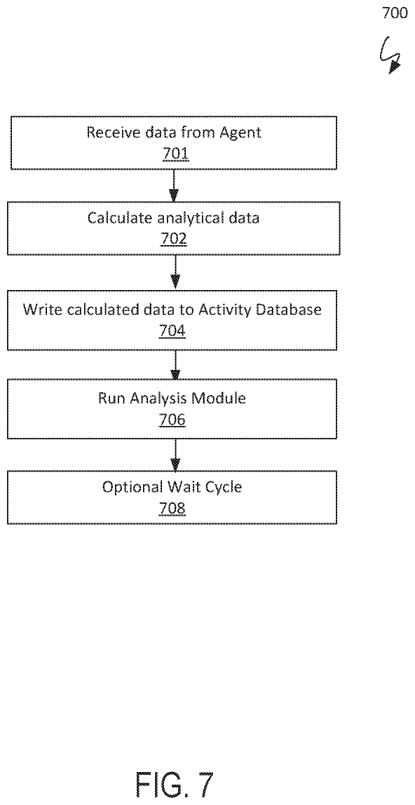

FIG. 7 is a flowchart illustrating an exemplary method 700 for evaluating safety thresholds. Method 700 may be performed by executing the safety threshold module 150 of the network environment 100. The process begins with receiving data from the agent 114 or the Wi-Fi AP 102 describing the activity or inactivity of one or more individuals at step 701. Calculating data, such as duration which a user has been absent from the Wi-Fi AP's 102 sensor range at step 702. The data calculated in step 702 may be written to the threshold safety activity database 152 at step 704. Loading and running the analysis module 156 software at step 706. Optionally, operating a timer or other waiting mechanism (e.g., a clock). In the alternative, the safety threshold module 150 may wait until new data is received from the agent 114 at step 708.

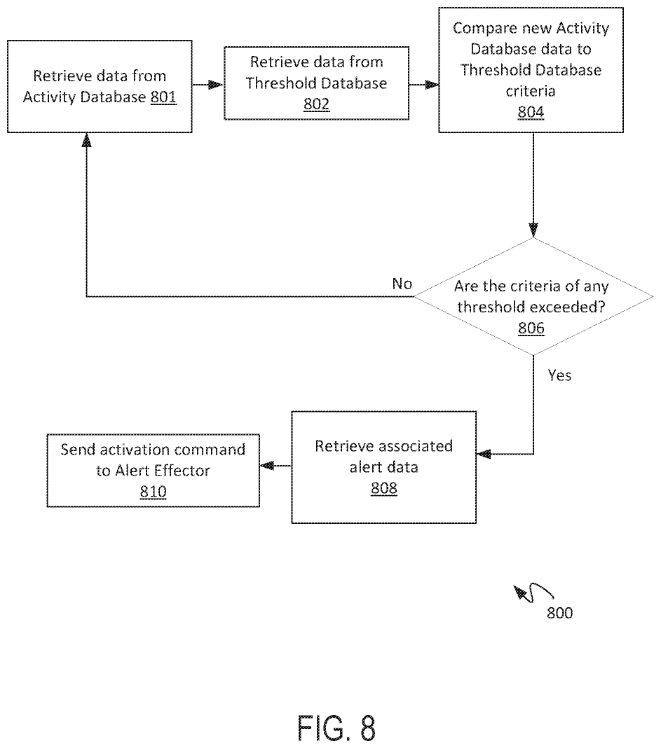

FIG. 8 is a flowchart illustrating an exemplary method 800 for condition analysis. Method 800 may be performed by executing the analysis module 156 of the safety threshold module 150. The process begins with retrieving all new data from the activity database 134 at step 801. The next step is retrieving all stored criteria from the threshold database 154 at step 802. The following step is comparing the data retrieved from the safety threshold activity database 152 with criteria retrieved from the threshold database 154 to determine if all the criteria of any threshold has been exceeded at step 804. Then the process determines if all the criteria of any threshold has been exceeded. If the threshold has been exceeded, proceed to step 808; if not, return to step 801 at step 806. The next step is retrieving from the threshold database 154, an alert effector ID and alert effector action associated with the exceeded threshold at step 808. The process ends by sending the alert effector action to the alert effector 158 for activation at step 810.

For clarity of explanation, in some instances the present technology may be presented as including individual functional blocks including functional blocks comprising devices, device components, steps or routines in a method embodied in software, or combinations of hardware and software. In some embodiments the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

Methods according to the above-described examples can be implemented using computer-executable instructions that are stored or otherwise available from computer readable media. Such instructions can comprise, for example, instructions and data which cause or otherwise configure a general purpose computer, special purpose computer, or special purpose processing device to perform a certain function or group of functions. Portions of computer resources used can be accessible over a network. The computer executable instructions may be, for example, binaries, intermediate format instructions such as assembly language, firmware, or source code. Examples of computer-readable media that may be used to store instructions, information used, and/or information created during methods according to described examples include magnetic or optical disks, flash memory, USB devices provided with non-volatile memory, networked storage devices, and so on.

Devices implementing methods according to these disclosures can comprise hardware, firmware and/or software, and can take any of a variety of form factors. Typical examples of such form factors include laptops, smart phones, small form factor personal computers, personal digital assistants, rackmount devices, standalone devices, and so on. Functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are means for providing the functions described in these disclosures.

Although a variety of examples and other information was used to explain aspects within the scope of the appended claims, no limitation of the claims should be implied based on particular features or arrangements in such examples, as one of ordinary skill could be able to use these examples to derive a wide variety of implementations. Further and although some subject matter may have been described in language specific to examples of structural features and/or method steps, it is to be understood that the subject matter defined in the appended claims is not necessarily limited to these described features or acts. For example, such functionality can be distributed differently or performed in components other than those identified herein. Rather, the described features and steps are disclosed as examples of components of systems and methods within the scope of the appended claims.

Claim language or other language reciting “at least one of” a set or “one or more of” a set” indicates that one member of the set or multiple members of the set (in any combination) satisfy the claim. For example, claim language reciting “at least one of A and B” means A, B, or A and B. In another example, claim language reciting “one or more of A and B” means A, B, or A and B. In another example, claim language reciting “one or more of A, B, and C” means A, B, C, A and B, A and C, B and C, or all of A, B, and C.

What is claimed is:

1. A method for Wi-Fi-based condition monitoring, the method comprising:
retrieving activity data regarding an individual in a monitored space, the activity data including channel state information (CSI) retrieved from a Wi-Fi access point located in proximity to the monitored space;
storing the retrieved activity data in an activity database that further stores past activity data regarding the individual;
polling a electronic medical records database for one or more health events each associated with a set of health parameters;
comparing the stored activity data with the polled health events to identify when the stored activity data matches a set of health parameters associated with an identified one of the health events; and
sending a notification over a communication network to a designated device associated with a caregiver based on the match.

2. The method of claim 1, further comprising identifying an activity trend based on the retrieved activity data and the past activity data, and storing the identified activity trend in the activity database.

3. The method of claim 1, wherein polling the electronic medical records database includes polling a caregiver database that stores a set of custom health parameters associated with at least one of the health events, and further comprising sending a second notification to the designated device based on a match between the stored activity data and the set of custom health parameters.

4. The method of claim 3, further comprising receiving one or more updates to the custom health parameters over the communication network from the designated device.

5. The method of claim 1, further comprising:
polling a threshold database that stores a plurality of sets of threshold parameters, each set associated with at least one of activity criteria, location change criteria, and time criteria;
comparing the stored activity data to the set of threshold parameters to determine that the stored activity data corresponds to at least one of the sets of threshold parameters; and
sending an alert effector action to an alert effector device based on the stored activity data corresponding to at least one of the sets of threshold parameters.

6. The method of claim 1, wherein the health events are associated with at least one of a diagnosis, a health condition, a procedure, or an operation.

7. The method of claim 1, wherein at least one of the health parameters include one of average rate of trips to a bathroom, sleep time, and active movement.

8. The method of claim 1, further comprising translating the activity data into a radio map based on Wi-Fi signal strengths at a plurality of locations within the space, the radio map indicating movement of the individual among the locations.

9. A non-transitory computer-readable storage medium having embodied thereon instructions executable by one or more processors to perform a method for Wi-Fi-based condition monitoring, the method comprising:
retrieving activity data regarding an individual in a monitored space, the activity data including channel state information (CSI) retrieved from a Wi-Fi access point located in proximity to the monitored space;
storing the retrieved activity data in an activity database that further stores past activity data regarding the individual;
polling a electronic medical records database for one or more health events each associated with a set of health parameters;
comparing the stored activity data with the polled health events to identify when the stored activity data matches a set of health parameters associated with an identified one of the health events; and
sending a notification over a communication network to a designated device associated with a caregiver based on the match.

10. The non-transitory computer-readable storage medium of claim 9, further comprising instructions executable to identify an activity trend based on the retrieved activity data and the past activity data, and to store the identified activity trend in the activity database.

11. The non-transitory computer-readable storage medium of claim 9, wherein polling the electronic medical records database includes polling a caregiver database that stores a set of custom health parameters associated with at least one of the health events, and further comprising instructions executable to send a second notification to the designated device based on a match between the stored activity data and the set of custom health parameters.

12. The non-transitory computer-readable storage medium of claim 11, further comprising instructions executable to receive one or more updates to the custom health parameters over the communication network from the designated device.

13. The non-transitory computer-readable storage medium of claim 9, further comprising instructions executable to:
poll a threshold database that stores a plurality of sets of threshold parameters, each set associated with at least one of activity criteria, location change criteria, and time criteria;
compare the stored activity data to the set of threshold parameters to determine that the stored activity data corresponds to at least one of the sets of threshold parameters; and
send an alert effector action to an alert effector device based on the stored activity data corresponding to at least one of the sets of threshold parameters.

14. The non-transitory computer-readable storage medium of claim 9, wherein the health events are associated with at least one of a diagnosis, a health condition, a procedure, or an operation.

15. The non-transitory computer-readable storage medium of claim 9, wherein at least one of the health parameters include one of average rate of trips to a bathroom, sleep time, and active movement.

16. The non-transitory computer-readable storage medium of claim 9, further comprising instructions executable to translate the activity data into a radio map based on Wi-Fi signal strengths at a plurality of locations within the space, the radio map indicating movement of the individual among the locations.

17. A system for Wi-Fi-based condition monitoring, the system comprising:
a Wi-Fi access point located in proximity to the monitored space; and
a cloud server that:
retrieves activity data from the Wi-Fi access point regarding an individual in the monitored space, the activity data including channel state information (CSI);
stores the retrieved activity data in an activity database that further stores past activity data regarding the individual;
polls a electronic medical records database for one or more health events each associated with a set of health parameters;
compares the stored activity data with the polled health events to identify when the stored activity data matches a set of health parameters associated with an identified one of the health events; and
sends a notification over a communication network to a designated device associated with a caregiver based on the match.

18. The system of claim 17, wherein the cloud server further identifies an activity trend based on the retrieved activity data and the past activity data, and stores the identified activity trend in the activity database.

19. The system of claim 17, wherein the cloud server polls the electronic medical records database by polling a caregiver database that stores a set of custom health parameters associated with at least one of the health events, and wherein the cloud server further sends a second notification to the designated device based on a match between the stored activity data and the set of custom health parameters.

20. The system of claim 17, wherein the cloud server further translates the activity data into a radio map based on Wi-Fi signal strengths at a plurality of locations within the space, the radio map indicating movement of the individual among the locations.
